SEQUENCE LISTING (21 sequences)

SEQ ID NO: 1 - protein, 6 aa, Artificial Sequence
  Synthetic derived from Oryza sativa
VGVAPG

SEQ ID NO: 2 - protein, 5 aa, Artificial Sequence
  Synthetic, wherein X is any amino acid
GXXPG

SEQ ID NO: 3 - protein, 6 aa, Artificial Sequence
  Synthetic peptide
XXXXXX

SEQ ID NO: 4 - protein, 6 aa, Artificial Sequence
  Synthetic derived from Oryza sativa
VGAMPG

SEQ ID NO: 5 - protein, 6 aa, Artificial Sequence
  Synthetic derived from Oryza sativa
VGLSPG

SEQ ID NO: 6 - protein, 6 aa, Artificial Sequence
  Synthetic derived from Oryza sativa
IGAMPG

SEQ ID NO: 7 - protein, 6 aa, Artificial Sequence
  Synthetic derived from Oryza sativa
IGLSPG

SEQ ID NO: 8 - protein, 17 aa, Artificial Sequence
  Synthetic derived from Oryza sativa
VGAMPGAAAAAVGAMPG

SEQ ID NO: 9 - protein, 17 aa, Artificial Sequence
  Synthetic derived from Oryza sativa
VGLSPGAAAAAVGLSPG

SEQ ID NO: 10 - protein, 17 aa, Artificial Sequence
  Synthetic derived from Oryza sativa
VGVAPGAAAAAVGVAPG

SEQ ID NO: 11 - protein, 17 aa, Artificial Sequence
  Synthetic derived from Oryza sativa
IGAMPGAAAAAIGAMPG

SEQ ID NO: 12 - protein, 17 aa, Artificial Sequence
  Synthetic derived from Oryza sativa
IGLSPGAAAAAIGLSPG

SEQ ID NO: 13 - protein, 6 aa, Artificial Sequence
  Synthetic derived from Oryza sativa
IGVAPG

SEQ ID NO: 14 - protein, 17 aa, Artificial Sequence
  Synthetic derived from Oryza sativa
IGVAPGAAAAAIGVAPG

SEQ ID NO: 15 - protein, 11 aa, Artificial Sequence
  Synthetic derived from human tropelastin
AKAAAKAAAKA

SEQ ID NO: 16 - protein, 6 aa, Artificial Sequence
  Synthetic peptide
XXXXXX

SEQ ID NO: 17 - protein, 7 aa, Artificial Sequence
  Synthetic peptide
XXXXXXX

SEQ ID NO: 18 - protein, 23 aa, Homo sapiens
GGTGCGGTGGTTCCTCAGCCTGG

SEQ ID NO: 19 - protein, 21 aa, Homo sapiens
GGGCCTTGAGATACCCCAGTG

SEQ ID NO: 20 - protein, 21 aa, Homo sapiens
GTTCCTGGTTCGCATCCTCCT

SEQ ID NO: 21 - protein, 21 aa, Homo sapiens
GCCAGGTAATGTCTGGATGGC

(19) United States
(12) Patent Application Publication
(10) Pub. No.: US 2012/0116346 A1
(43) Pub. Date: May 10, 2012

(54) HYPERTHERMIA INDUCED DEPOSITION OF ELASTIN FIBERS

(76) Inventors: Thomas F. MITTS, Visalia, CA (US); Aleksander Hinek, Toronto (CA)

(21) Appl. No.: 13/289,921

(22) Filed: Nov. 4, 2011

Related U.S. Application Data
(60) Provisional application No. 61/410,855, filed on Nov. 5, 2010.

(51) Int. Cl. A61M 37/00; A61F 7/00

(52) U.S. Cl. 604/500; 607/96

(57) ABSTRACT

Methods for inducing the deposition of elastin at a tissue site by exposing the tissue to hyperthermia is described herein.

### CROSS-REFERENCE TO RELATED APPLICATIONS

This application claims priority to U.S. Provisional Patent Application No. 61/410,855, entitled “HYPERTHERMIA INDUCED DEPOSITION OF ELASTIN FIBERS” filed Nov. 5, 2010, which is herein incorporated by reference in its entirety.

### GOVERNMENT INTERESTS

This invention was partially funded by a grant from the Canadian Institute of Health Research (grant No. PG 13920) and the Heart and Stroke Foundation of Ontario (grant No. NA 4381).

### PARTIES TO A JOINT RESEARCH AGREEMENT

Not Applicable

### INCORPORATION BY REFERENCE OF MATERIAL SUBMITTED ON A COMPACT DISC

Not Applicable

### BACKGROUND

Not applicable

### BRIEF SUMMARY OF THE INVENTION

Embodiments presented herein are directed to methods of improving the appearance of a tissue site in a subject in need thereof comprising exposing the tissue to hyperthermia. In one embodiment, hyperthermia may comprise a temperature of about 39° C. to about 41° C. In yet another embodiment, the temperature may be about 39° C., about 39.5° C., about 40° C., about 40.5° C. and about 41° C.

Embodiments presented herein are directed to methods of improving the appearance of a tissue site in a subject in need thereof comprising applying heat to the tissue site. In one embodiment, hyperthermia may comprise a temperature of about 39° C. to about 41° C. In yet another embodiment, the temperature may be about 39° C., about 39.5° C., about 40° C., about 40.5° C. and about 41° C. In one embodiment, the tissue site may comprise scar tissue, visible lines, wrinkles and a combination thereof. In one embodiment, the subject may suffer from elastinopathy. In one embodiment, hyperproliferative collagenous neointimal formation in arterial SMC is inhibited. In yet another embodiment, the production of insoluble elastin in the tissue to which it is administered is stimulated. In yet another embodiment, the endogenous synthesis and deposition of elastin in the tissue to which it is administered is stimulated. In yet another embodiment, the deposition of collagen in the tissue to which it is administered is stimulated and the appearance of a site presenting visible lines or wrinkles is improved. In one embodiment, the appearance of a site comprising scar tissue is improved. In another embodiment, net deposition of chondroitin-sulfate containing moieties is inhibited. In one embodiment, the production of collagen is stimulated.

In yet another embodiment, the tissue site may comprise scar tissue, visible lines, wrinkles and combinations thereof. In another embodiment, the subject suffers from elastinopathy. The elastinopathy may comprise Marfan syndrome; autosomal dominant Weill-Marchesani syndrome; severe neonatal Marfan syndrome; dominant ectopia lentis; isolated skeletal features of Marfan syndrome; Beals syndrome; Familial mitral valve prolapse syndrome (MVP); mitral valve prolapse, myopia, minimal or no aortic dilation, subtle skeletal changes and skin changes (MASS phenotype); Shprintzen-Goldberg syndrome; supravalvular aortic stenosis; Williams syndrome; Costello syndrome, Loeys-Dietz syndrome and Cutis laxa.

In one embodiment, the method may further comprise administering an agent selected from epitopes, cytokines, growth factors and combinations thereof. In one embodiment, the method may further comprise administering retinoic acid. In one embodiment, the method may further comprise administering an elastogenic peptide. In various embodiments, the elastogenic peptide may be administered by injection or topical administration. In yet another embodiment, the method may further comprise administering an effective amount of an elastogenic peptide. The elastogenic peptide may be VGVAPG (SEQ ID NO. 1), VGAMPG (SEQ ID NO. 4), VGLSPG (SEQ ID NO. 5), IGAMPG (SEQ ID NO. 6), IGLSPG (SEQ ID NO. 7), VGAMPGAAAAAVGAMPG (SEQ ID NO. 8), VGLSPGAAAAAVGLSPG (SEQ ID NO. 9), VGVAPGAAAAAVGVAPG (SEQ ID NO. 10), IGAMPGAAAAAIGAMPG (SEQ ID NO. 11), IGLSPGAAAAAIGLSPG (SEQ ID NO. 12), IGVAPG (SEQ ID NO. 13), IGVAPGAAAAAIGVAPG (SEQ ID NO. 14) and combinations thereof.

In one embodiment, the tissue site may comprise connective tissue. In yet another embodiment, the tissue is selected from the group consisting of dermal fibroblasts, smooth muscles cells, mouth tissue, hair follicles, and corneal tissue.

### DESCRIPTION OF DRAWINGS

The file of this patent contains at least one photograph or drawing executed in color. Copies of this patent with color drawing(s) or photograph(s) will be provided by the Office upon request and payment of necessary fee.

For a fuller understanding of the nature and advantages of the present invention, reference should be made to the following detailed description taken in connection with the accompanying drawings.

FIG. 1. Cultures of normal human skin fibroblasts and normal human aortic SMCs maintained for five days at mildly hyperthermic temperatures (41 and 39° C.) produce more elastic fibers than cultures kept at 37° C. (A) Representative micrographs depicting immunodetection of elastic fibers in five-day-old cultures of normal dermal fibroblasts and normal aortic SMCs maintained at 37 and 41° C. Scale bar=15 μm. (B) Results from the quantitative analysis of the indicated ECM components immunodetected in parallel cultures demonstrate that hyperthermia exclusively upregulated the deposition of elastic fibers and downregulated the deposition of chondroitin-sulfate-containing moieties. (C) Results from the quantitative biochemical assay of cross-linked (insoluble) elastin after metabolic labeling of cultured cells with [³H]-valine, demonstrating a time course increase in the deposition of elastic fibers in hyperthermia-exposed cultures. (D) Five-day-old cultures of both cell types exposed to 39° C. also demonstrated a significant upregulation in the net deposition of [³H]-valine-labeled insoluble elastin. The parallel cultures exposed to 41 or 39° C. for only three hours a day also produced more insoluble elastin than cultures kept at 37° C.

FIG. 2. Hyperthermia upregulates the production of elastic fibers in stretch-marked skin. (A) Representative micrographs depicting elastic fibers in seven-day-old cultures of dermal fibroblasts isolated from the biopsy of stretch-marked skin from a 36-old female patient. Scale bar=15 μm. (B) Results of morphometric evaluation of elastic fibers and quantitative assay of metabolically labeled insoluble elastin demonstrate that cultures of stretch mark-derived fibroblasts maintained at 41° C. produced more elastic fibers than parallel cultures maintained at 37° C. (C) Representative micrographs depicting black elastic fibers (detected with Movat's method) in transverse sections of cultured skin explants derived from the stretch-marked skin of a 26-year old female. Scale bar=30 μm. (D) Left panel: results of morphometric evaluation of elastic fibers detected with Movat's stained sections of the initial biopsies (Bps) and of these biopsy-derived explants maintained for seven days at 37 or 41° C. Right panel: levels of metabolically labeled insoluble elastin detected in explants of stretch-marked skin cultured for seven days at 37 or 41° C.

FIG. 3. Hyperthermia does not affect the steady-state level of tropoelastin and S-Gal/EBP mRNAs, but transiently enhances the intracellular levels of these two proteins and then stimulates their intracellular association and joint secretion. (A) Results of quantitative RT-PCR analysis demonstrating that a 24-hour-long exposure of dermal fibroblasts and aortic SMCs to hyperthermia did not affect elastin mRNA levels. (B) Western blots with an antibody recognizing only soluble tropoelastin detected more full-length (72 kDa) tropoelastin and less lower molecular weight degradation products in cells exposed to 41° C. for only three hours, as compared to their counterparts kept at 37° C. (C) Cells exposed to 41° C. (pulsed for three hours with [³H]-valine) secreted significantly more [³H]-valine-labeled tropoelastin (immunoprecipitated from the conditioned media) following a three-hour chase than cultures kept at 37° C. (D) Cells exposed to hyperthermia did not demonstrate any increase in the steady-state levels of S-Gal/EBP mRNA. (E) Western blotting revealed increased protein levels of this tropoelastin chaperone. (F) Representative micrographs showing a double immunostaining with antibodies against S-Gal/EBP (red fluorescence) and tropoelastin (green fluorescence) confirmed that hyperthermia enhanced the intracellular levels of these two proteins and their association (yellow fluorescence) in the perinuclear endosomal compartment and in peripheral secretory vesicles. (G) Representative micrographs depicting parallel cultured aortic SMCs immunostained with anti-FKBP65 (red fluorescence) and with anti-tropoelastin (green fluorescence) show that FKBP65 does not colocalize with intracellular or extracellular elastin. Scale bars=5 μm.

FIG. 4. Hyperthermia induces faster recycling of the S-Gal/EBP. In order to trace S-Gal/EBP trafficking, multiple subconfluent cultures of aortic SMCs were cooled to 4° C. and externally labeled with anti-S-Gal antibody for 15 minutes and then transferred either to 37 or 41° C. for the indicated periods of time. Representative micrographs obtained by fluorescence microscopy show that, five minutes after the transfer of cells to 37° C., the anti-S-Gal-labeled EBP molecules were still detected on the cell surface. They began internalization during the next ten minutes and could be clearly detected in the endocytic vesicles and in the endosomal cisternae localized in the cell center 15 minutes after transferring the cell to 37° C. The EBP/anti-S-Gal complexes were concentrated mostly in the perinuclear endosomal compartments (25 minutes after transferring the cell to 37° C.); following the next three ten-minute intervals, the anti-S-Gal/EBP complexes were present in the peripheral endosomal cisternas, in exocytic vesicles, and, again, at the cell surface (left panels). The overall EBP recycling process occurred significantly faster in cells maintained at 41° C., resulting in a second round of S-Gal/EBP recycling 55 minutes following the transfer of cells to 41° C. (right panels). Scale bar=5 μm.

FIG. 5. Hyperthermia induces the inhibition of chondroitin-6-sulfate deposition and the consecutive recovery of normal elastogenesis in cultures of CS fibroblasts. (A) CS fibroblasts maintained for three hours at 41° C. displayed four times more intracellular [³H]-valine-labeled tropoelastin and eventually secreted three times more of this metabolically labeled protein than their counterparts kept at 37° C. (B) Representative micrographs depicting five-day-old cultures of CS fibroblasts immunostained with anti-elastin antibody show the recovery of normal elastogenesis in hyperthermia-exposed cultures of CS fibroblasts. Scale bar=15 μm. (C) Results of quantitative assays of metabolically labeled insoluble elastin in five-day-old cultures of CS fibroblasts confirmed these results and additionally showed that CS fibroblasts heated for only three hours/day also demonstrated significantly higher levels of newly produced insoluble elastin than their counterparts maintained at 37° C. (D) Representative micrographs depicting five-day-old cultures of CS fibroblasts immunostained with anti-chondroitin sulphate and anti-chondroitin-6-sulfate antibodies. CS-derived fibroblasts exposed to 41° C. demonstrated strikingly lower content of both immunodetected chondroitin sulfates than cells maintained at 37° C. Scale bar=10 μm. (E) The 24 hour-long exposure of normal and CS-derived fibroblasts to 41° C. significantly inhibited the synthesis of [³⁵S]-sulphate-labeled glycosaminoglycans that could be immunoprecipitated with antibodies recognizing total chondroitin sulfate and chondroitin-6-sulfate, as compared to counterparts kept at 37° C. (F) Western blot analysis further showed that the heat-exposed CS fibroblasts demonstrated a marked increase in cell-associated S-Gal/EBP and tropoelastin, which occurred without any increase in their respective mRNA levels. (G) Representative micrographs showing 24-hour cultures of CS-derived fibroblasts (double immunostained with anti-EBP and anti-tropoelastin antibodies) demonstrate that the hyperthermia-exposed cells contain more secretory vesicles (yellow fluorescence), in which the immunodetected EBP (red fluorescence) colocalizes with the immunodetected tropoelastin (green fluorescence). Scale bar=5 μm.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

It must be noted that as used herein, and in the appended claims, the singular forms “a,” “an,” and “the” include plural reference unless the context clearly dictates otherwise. Thus, for example, reference to a “fibroblast” is a reference to one or more fibroblasts and equivalents thereof known to those skilled in the art.

As used herein, all claimed numeric terms are to be read as being preceded by the term, “about,” which means plus or minus 10% of the numerical value of the number with which it is being used. Therefore, a claim to “50%” means “about 50%” and encompasses the range of 45%-55%.

“Administering” when used in conjunction with a therapeutic means to administer a therapeutic directly into or onto a target tissue, or to administer a therapeutic to a patient whereby the therapeutic positively impacts the tissue to which it is targeted. “Administering” a composition may be accomplished by any mode including parenteral administration including injection, oral administration, topical administration, pleural infusion, pericardial infusion, or by any other method known in the art including for example electrical deposition (e.g., iontophoresis) and ultrasound (e.g., sonophoresis). In certain embodiments, the compositions described herein may be administered in combination with another form of therapy, including for example radiation therapy, infrared therapy, ultrasound therapy, or any other therapy know in the art or described herein.

In certain embodiments, the compositions may be combined with a carrier. A “carrier” as used herein may include, but is not limited to, an irrigation solution, antiseptic solution, other solution time released composition, elution composition, bandage, dressing, colloid suspension (e.g., a cream, gel, or salve) internal or external dissolvable sutures, dissolvable beads, dissolvable sponges and/or other materials or compositions known now or hereafter to a person of ordinary skill in the art.

The term “animal” as used herein includes, but is not limited to, humans and non-human vertebrates, such as wild, domestic, and farm animals.

The term “improves” is used to convey that the present invention changes either the appearance, form, characteristics and/or the physical attributes of the tissue to which it is being provided, applied or administered. The change in form may be demonstrated by any of the following, alone or in combination: enhanced deposition of elastin, increased elasticity of the tissue, reduced scar tissue formation or any other such improvement recognized in the art or described herein.

The term “inhibiting” includes the administration of a compound of the present invention to prevent the onset of the symptoms, alleviating the symptoms, or eliminating the disease, condition or disorder.

By “pharmaceutically acceptable,” it is meant that the carrier, diluent or excipient must be compatible with the other ingredients of the formulation and not deleterious to the recipient thereof. By “excipient,” it is meant any inert or otherwise non-active ingredient, which can be added to the active ingredient which may improve the overall composition's properties, such as improving shelf-life, improving retention time at the application site, improving flowability, improving consumer acceptance, et alia.

As used herein, the term “therapeutic” means an agent utilized to treat, combat, ameliorate, prevent or improve an unwanted condition or disease of a patient.

A “therapeutically effective amount” or “effective amount” of a composition is a predetermined amount calculated to achieve the desired effect, i.e., to increase production of elastin or the deposition of elastic fibers. For example, a therapeutic effect may be demonstrated by increased elastogenesis, increased cellular proliferation, increased digestion or resorption of scar material, reduction of symptoms and sequellae as well as any other therapeutic effect known in the art. The activity contemplated by the present methods includes both medical therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, the physical characteristics of the patient (height, weight, etc.), and the condition being treated. It will be understood that the effective amount administered will be determined by the physician in light of the relevant circumstances, including the condition to be treated, the choice of compound to be administered, and the chosen route of administration, and therefore, the dosage ranges provided are not intended to limit the scope of the invention in any way. A “therapeutically effective amount” of compound of this invention is typically an amount such that when it is administered in a physiologically tolerable excipient composition, it is sufficient to achieve an effective systemic concentration or local concentration in the tissue.

The terms “treat,” “treated,” or “treating” as used herein refers to both therapeutic treatment and prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects.

Generally speaking, the term “tissue” refers to any aggregation of similarly specialized cells which are united in the performance of a particular function. As used herein, “tissue,” unless otherwise indicated, refers to tissue which includes elastin as part of its necessary structure and/or function. For example, connective tissue which is made up of, among other things, collagen fibrils and elastin fibrils satisfies the definition of “tissue” as used herein. Additionally, elastin appears to be involved in the proper function of blood vessels, veins, and arteries in their inherent visco-elasticity.

The extracellular matrix (ECM) is made up of fibronectin, laminin, collagen and elastic fibers, as well as numerous glycosaminoglycans and protoglycans. These ECM components are organized into a network of rope-like structures which underlies many tissues, such as, blood vessels, skin, tendons, ligaments, and lungs. Of these ECM components, elastin is unique in that it can be stretched to over 150 percent of its original length and rapidly returns to its original size and shape. This property provides tissues in which elastin is incorporated with the ability to resume their original form after stretching. Therefore, elastin and elastin fibers allow these tissues to maintain the resiliency, stretchability and shape of these tissues.

Elastic fiber formation (elastogenesis) is a complex process involving intracellular and extracellular events. Cells such as fibroblasts, endothelial cells, chondroblasts or vascular smooth muscle cells, first synthesize and secrete glycoproteins that form a microfibrillilar scaffold into the extracellular space. Tropoelastin, the soluble precursor peptide of elastin, is synthesized in these cells by ribosomes in the rough endoplasmatic reticulum and transported through the Golgi apparatus and secretory vesicles that deposit tropoelastin in the extracellular space. Once outside the cell, tropoelastin is assembled into long chains and covalently cross-linked by lysyl oxidase. During crosslinking, unique composite amino acids, desmosine and isodesmosine, which join the tropoelastin chains, are formed and insoluble elastin is created.

Elastin fibers are composed of two major components: an amorphous, elastin core which makes up the bulk (>90%) of the fiber; and the 10-12 nm microfibrilary component surrounding the elastin core made up of glycoproteins, such as, for example, fibrillins, fibulins and microfibril-associated glycoproteins (MAGPs). Elastin may also be interwoven with non-elastic collagen fibers to limit stretching and prevent tearing of certain tissues. Mature (insoluble) elastin is metabolically inert and remains the most durable element of extracellular matrix. In undisturbed tissues, mature elastin may last for the lifetime of the tissue.

Deposition of elastin in the ECM appears to be controlled on both the transcriptional level (tropoelastin mRNA message expression) and post-transcriptional level (tropoelastin message stability). Other post-transcriptional events which control secretion of tropoelastin monomers, extracellular assembly of tropoelastin, and regulation of cross-linking of tropoelastin may also control elastin deposition.

The proper mechanical performance of the myocardium depends on the contractile properties of cardiac myocytes that are supported by the mechanical strength and resiliency of the extracellular matrix (ECM). Following myocardial injury, the cardiac ECM undergoes dynamic local remodeling, which leads to the production of scar tissue. However, overzealous ECM production in postinfarct hearts may lead to maladaptive fibrosis and contribute to heart failure.

Elastic fibers constitute the major fibrotic component of the extracellular matrix (ECM) and are responsible for the resilience of blood vessels, lungs, skin, and the connective tissue framework of internal organs. They are composed of a microfibrillar scaffold made up of several glycoproteins and a core consisting of the unique protein polymer elastin. Elastin is formed after the lysyl-oxidase-catalyzed cross-linking of multiple precursor molecules (tropoelastin) that are produced and secreted by fibroblasts, chondroblasts, and vascular smooth muscle cells (SMCs). Elastogenesis is also modulated by a 67 kDa elastin binding protein (EBP), identified as the catalytically inactive spliced variant of β-galactosidase (S-Gal) that has retained the ability to bind to galactosugars and acquired a unique (frame-shift-encoding) elastin-binding domain. The 67 kDa S-Gal/EBP serves as a molecular chaperone for intracellular tropoelastin, which binds this highly hydrophobic and unglycosylated protein and escorts it through the secretory pathways, protecting it from premature self-aggregation and proteolytic degradation and assuring its orderly assembly into elastic fibers.

Embodiments of the present invention are directed to methods and processes of coordinated dissociation of tropoelastin from its chaperone and its consecutive assembly into elastic fibers after the binding of S-Gal/EBP to galactosylated components of the microfibrillar scaffold.

Embodiments of the present invention are also directed to a finding that S-Gal/EBP molecules (40 to 50 percent) recycle back to the cell interior following delivery of tropoelastin to the cell surface. These chaperone molecules bind again to their new tropoelastin partners in the recycling endosomes and escort them to the cell surface in consecutive rounds.

The primary genetic deficiency of the S-Gal/EBP occurring in patients with GMi-gangliosidosis or Morquio B syndrome, or the secondary deficiency of this tropoelastin chaperone—due to the constant shedding induced by its abnormal accumulation of chondroitin-6-sulfate (CS) or dermatan sulfate glycosaminoglycans (Hurler disease)—prevents the normal assembly of elastic fibers and contributes to the development of the severe clinical phenotypes of these syndromes. Importantly, the genetic manipulations leading to the experimental elimination of proteoglycans rich in chondroitin-6-sulfate and dermatan sulfate (versicans 1 and 2 or biglycan) leads to the rescue of S-Gal/EBP and to the restoration of normal elastogenesis in human and animal cells.

The results of the present studies involving normal human aortic SMCs and dermal fibroblasts derived from normal human skin, stretch-marked human skin of adult patients, and wrinkled skin of children with CS demonstrate for the first time that exposure to mild hyperthermia inhibits the deposition of chondroitin-sulfate-containing moieties, which are associated with a significant net upregulation in the deposition of elastic fibers but not collagen I or fibronectin. In addition to inhibiting chondroitin-6-sulfate-containing moieties that lead to the preservation of S-Gal/EBP molecules, hyperthermia also induces their faster recycling. This, in turn, triggers a more efficient preservation of newly synthesized tropoelastin, enhancement of its secretion and extracellular assembly into elastic fibers.

Embodiments are directed to methods of improving the appearance of a tissue site in a subject in need thereof comprising applying heat to the tissue site. For example, the heat applied to said tissue site may comprise a temperature of about 39° C. to about 41° C., including, for example, about 39° C., about 39.5° C., about 40° C., about 40.5° C. and about 41° C.

In embodiments, the tissue site may comprise scar tissue, visible lines, wrinkles and combinations thereof. In embodiments, the tissue site may comprise connective tissue. In embodiments, the tissue may include, dermal fibroblasts, smooth muscles cells, mouth tissue, hair follicles, and corneal tissue.

In embodiments, the subject may suffer from elastinopathy, wherein the elastinopathy is treated by the methods described herein. The elastinopathy may comprise Marfan syndrome; autosomal dominant Weill-Marchesani syndrome; severe neonatal Marfan syndrome; dominant ectopia lentis; isolated skeletal features of Marfan syndrome; Beals syndrome; Familial mitral valve prolapse syndrome (MVP); mitral valve prolapse, myopia, minimal or no aortic dilation, subtle skeletal changes and skin changes (MASS phenotype); Shprintzen-Goldberg syndrome; supravalvular aortic stenosis; Williams syndrome; Costello syndrome, Loeys-Dietz syndrome and Cutis laxa.

In embodiments, the net deposition of chondroitin-sulfate containing moieties is inhibited. In embodiments, hyperproliferative collagenous neointimal formation in arterial SMC is inhibited. In embodiments, the production of insoluble elastin in the tissue site to which it is administered is stimulated. In embodiments, the endogenous synthesis and deposition of elastin in the tissue site to which it is administered is stimulated. In embodiments, the deposition of collagen in the tissue site to which it is administered is stimulated. In embodiments, the appearance of the tissue site presenting visible lines or wrinkles is improved. In embodiments, the appearance of the tissue site comprising scar tissue is improved. In embodiments, the production of collagen is stimulated.

Embodiments are directed to methods of improving the appearance of a tissue site in a subject in need thereof comprising applying heat to the tissue site. The tissue is heated to achieve hyperthermia. In embodiments of the present invention, hyperthermia may be achieved when the target tissue reaches a temperature of from about 39° C. to about 41° C., from about 39° C. to about 40° C., from about 40° C. to about 41° C., from about 39.5° C. to about 41.5° C., from about 39.5° C. to about 40.5° C., from about 40.5° C. to about 41.5° C., about 39° C., about 39.5° C., about 40° C., about 40.5° C. about 41° C., and about 41.5° C.

In embodiments of the present invention, hyperthermia may be achieved by indirectly or directly apply a heat source or radiant heat to the tissue being targeted. In certain embodiments, heat can be provided by an energy source, such as, but not limited to, microwave, radiofrequency and ultrasound. In another embodiment, hyperthermia is achieved with hydrocollator packs, heat masks, heat packs, hot water, ultrasound or other devices that provide heat and combinations thereof. In yet another embodiment, hyperthermia is achieved by administration a compound locally that results in heating a tissue locally or systematically. In embodiments, various devices or techniques are used to provide or apply heat to achieve hyperthermia. Non-limiting examples of heating techniques or devices are masks, heating wraps, lamps, lasers and combinations thereof. In yet another example, natural, pharmaceutical, and combinations are applied to the subject as a source of heat.

In embodiments, the subject is exposed to heat at various lengths of time. For example, in one embodiment, the subject is exposed to heat for about 5 minutes to about 1 hour. In yet another embodiment, the subject is exposed to heat for, but not limited to, about 5 minutes, about 10 minutes, about 15 minutes, about 20 minutes, about 25 minutes, about 30 minutes, about 35 minutes, about 40 minutes, about 45 minutes, about 50 minutes, about 55 minutes and about 1 hour.

In embodiments, the subject is exposed to heat at different frequencies. For example, in one embodiment, the subject is exposed to heat once a day, once every 2 days, once every 3 days, once every 4 days, once every 5 days, once every 6 days or once a week. In embodiments, the subject may be exposed to heat one to three times a day, including once a day, twice a day and three times a day.

In certain embodiments, the tissue site may comprise scar tissue, visible lines, wrinkles and combinations thereof.

Embodiments presented herein are directed to methods for increasing the net deposition of elastin at a tissue site comprising exposing the tissue to hyperthermia in a subject in need thereof.

Embodiments of the present invention are directed to methods for increasing the net deposition of elastin at a tissue site wherein the tissue site comprises scar tissue, visible lines, wrinkles and combinations thereof.

In one embodiment, the net deposition of chondroitin-sulfate containing moieties is inhibited in a tissue when the tissue is exposed to hyperthermia.

In certain embodiments, the subject may be suffering from elastinopathy. In one embodiment, elastinopathy comprises Marfan syndrome; autosomal dominant Weill-Marchesani syndrome; severe neonatal Marfan syndrome; dominant ectopia lentis; isolated skeletal features of Marfan syndrome; Beals syndrome; Familial mitral valve prolapse syndrome (MVP); mitral valve prolapse, myopia, minimal or no aortic dilation, subtle skeletal changes and skin changes (MASS phenotype); Shprintzen-Goldberg syndrome; supravalvular aortic stenosis; Williams syndrome; Costello syndrome, Loeys-Dietz syndrome and Cutis laxa. In certain embodiments, the elastinopathy may be treated.

In embodiments, the method may further comprise administering an effective amount of an elastogenic peptide. Elastogenic peptides may be administered, for example, by injection or topical administration. In embodiments, the method may further comprise administering an effective amount of an elastogenic peptide selected from VGVAPG (SEQ ID NO. 1), VGAMPG (SEQ ID NO. 4), VGLSPG (SEQ ID NO. 5), IGAMPG (SEQ ID NO. 6), IGLSPG (SEQ ID NO. 7), VGAMPGAAAAAVGAMPG (SEQ ID NO. 8), VGLSPGAAAAAVGLSPG (SEQ ID NO. 9), VGVAPGAAAAAVGVAPG (SEQ ID NO. 10), IGAMPGAAAAAIGAMPG (SEQ ID NO. 11), IGLSPGAAAAAIGLSPG (SEQ ID NO. 12), IGVAPG (SEQ ID NO. 13), IGVAPGAAAAAIGVAPG (SEQ ID NO. 14) and combinations thereof.

U.S. patent application Ser. No. 10/778,253, filed on Feb. 13, 2004 entitled, “Elastin Digest Compositions and Methods Utilizing Same,” herein incorporated by reference, describes various compositions for the therapeutic and/or cosmetic treatment of elastin comprising tissues. Preferably such compositions stimulate the endogenous production of elastin or appear to enhance the elasticity of the skin and provide an external supply of peptide precursors of elastin that penetrate into the tissue to which it is applied. The Application describes compositions comprising an elastin digest derived from proteolytic digestion of insoluble elastin derived from mammalian ligaments with a protein digesting composition. Such compositions and techniques may be used in the various methods as described herein to treat stretch marked or otherwise damaged or aged skin.

For example, suitable compositions according to U.S. patent application Ser. No. 10/778,253 include, commercially available, Elastin E91 preparation from Protein Preparations, Inc., St. Louis, Mo., is a suitable elastin product to subject to digestion, having about 1,000 to 60,000 dalton molecular weight. Additionally, a series of digests available under the trade name ProK, and specifically ProK60, are elastin peptide mixture derived from the proteolytic digestion of insoluble Elastin derived from bovine neck ligaments, commercially available from Human Matrix Sciences, LLC. The digestion is accomplished with Proteinase K enzyme. The commercially available products will be referred to as E91 and ProK respectively and may be employed in the present therapeutic treatments described herein relating to stretch marked skin.

The term as used herein, “elastin digest” refers to any insoluble elastin derived from mammalian tissue or previously solubilized elastin (either chemically or enzymatically) that is proteolytically digested with a protein digesting composition. As described in the U.S. application Ser. No. 10/778,253, an elastin digest is a mixture of peptides. Additionally, the elastin digest of the present invention may comprise other epitopes for extracellular matrix proteins, cytokines, growth factors, and di-peptides.

Suitable elastin digests may be obtained from proteolytic digestion, with a protein digesting composition, of insoluble elastin derived from connective mammalian tissues or ligaments, bovine neck ligaments in particular. Suitable protein digesting compositions, include for example, human elastase enzyme, Proteinase K enzyme, and thermolysin. Other suitable elastinogenic peptides may comprise plant-derived elastogenic peptides.

Exemplary plant-derived elastogenic peptides are described in U.S. Pat. No. 7,723,308 entitled “Plant-Derived Elastin Binding Protein Ligands and Methods of Using the Same” filed Apr. 17, 2006, herein incorporated by reference in its entirety, discloses peptides, or peptide mimetics thereof, comprising the formula X₁GX₂X₃PG wherein X₁ is the amino acid V or I; X₂ is the amino acid A, L or V; and X₃ is the amino acid M, S, or A may be provided, which bind to the elastin receptor on cells and stimulate the endogenous production of elastin-enriched ECM. The sextapeptide may be supplemented with one or more additional linking amino acid residues, for example the linking amino acid residues may comprise alanine residues, or analogues thereof. The linking amino acid residues link two sextapeptides, or peptide mimetics thereof, generating a sextapeptide dimer. The two sextapeptides, or peptide mimetics thereof, within a linked sextapeptide dimer may be identical. Alternatively, the two sextapeptides, or peptide mimetics thereof, within a linked sextapeptide dimer may be different. The number of linking residues linking two sextapeptides, or peptide mimetics thereof, may be in the range of about 3-7, more preferably in the range from about 4-5.

The elastin peptide fragment component in a therapeutic formulation is typically present in amount from about 0.0002 to about 90% by weight of the formulation. These formulations may be employed directly as a constituent of therapeutic treatment, such as emulsions, lotions, sprays, ointments, creams and foam masks. Final products may contain up to 10% by weight but preferably 0.001 to 5% of such a solution though of course more concentrated or more dilute solutions may also be used in greater or lesser amounts. For example, an eye cream may comprise about 0.1% (w/w) and a facial cream may comprise about 0.025% (w/w) of a soluble elastin peptide fragment component in an excipient. Facial cream compositions usually comprise salts. Specifically, the elastin peptide fragment component of the present invention exists in cosmetic or therapeutic compositions at concentrations of about 10-1000 μg/ml, preferably about 25 μg/ml.

The protein motif VGVAPG (SEQ ID NO. 1) has been previously shown to stimulate proliferation/migration of monocytes, dermal fibroblasts, and smooth muscle cells through its interaction with the cell-surface elastin receptor. Other GXXPG (SEQ ID NO. 2) sequences recognized by BA4 antibody are also known ligands for the elastin receptor. More recently, it has been shown that elastin peptides, liberated through proteolytic digestion of bovine ligamentum nuchae and containing elastin receptor ligand sequences (GXXPG) (SEQ ID NO. 2) also induce elastogenesis in dermal fibroblasts through interaction with the elastin receptor.

In various embodiments, the sextapeptide comprises the sequence X₁-X₂-X₃-X₄-X₅-X₆ (SEQ ID NO. 3), wherein X₁ is V or I, X₂ is G, X₃ is A or L, X₄ is M or S, X₅ is P and X₆ is G. In further embodiments, a linked sextapeptide is provided comprising one or more linking amino acid residues wherein the linking residues join two sextapeptide compounds, each sextapeptide having the sequence X₁-X₂-X₃-X₄-X₅-X₆ (SEQ ID NO. 3), wherein X₁ is V or I, X₂ is G, X₃ is A or L, X₄ is M or S, X₅ is P and X₆ is G. In embodiments, the sextapeptide of the invention comprises VGAMPG (SEQ ID NO. 4), VGLSPG (SEQ ID NO. 5), IGAMPG (SEQ ID NO. 6), or IGLSPG (SEQ ID NO. 7). In embodiments, a linked sextapeptide (or sextapeptide dimer) of the invention comprises VGAMPGAAAAAVGAMPG (SEQ ID NO. 8), VGLSPGAAAAAVGLSPG (SEQ ID NO. 9), VGVAPGAAAAAVGVAPG (SEQ ID NO. 10), IGAMPGAAAAAIGAMPG (SEQ ID NO. 11), or IGLSPGAAAAAIGLSPG (SEQ ID NO. 12).

In another embodiment, a sextapeptide that comprises the sequence IGVAPG (SEQ ID NO. 13) may be used. In one embodiment, a linked sextapeptide that comprises the sequence IGVAPGAAAAAIGVAPG (SEQ ID NO. 14) may be used. Another linked sextapeptide of the invention comprises the sequence of two sextapeptides having the sequence IGVAPG (SEQ ID NO. 13) that are joined by a linker may be used.

In a further embodiment, the linking moiety can be any moiety recognized by those skilled in the art as suitable for joining the sextapeptides so long as the sextapeptide compound(s) retain the ability to interact with the elastin receptor and induce elastogenesis. The linking moiety may be comprised of, for example, but not limited to, at least one of alanine or any other amino acid, a disulfide bond, a carbonyl moiety, a hydrocarbon moiety optionally substituted at one or more available carbon atoms with a lower alkyl substituent. Optimally, the linking moiety is a lysine residue or lysine amide, i.e., a lysine residue wherein the carboxyl group has been converted to an amide moiety —CONH.

Non-peptide or partial peptide mimetics of any of the aforementioned synthetic peptides may be employed in the embodiments described herein The compound may have the formula X₁-X₂-X₃-X₄-X₅-X₆ (SEQ ID NO. 16), wherein X₁ is V or I or a mimetic of V or I; X₂ is G, or a mimetic of G; X₃ is A or L, or a mimetic of A or L; X₄ is M or S, or a mimetic of M or S, X₅ is P or a mimetic of P and X₆ is G or a mimetic of G. In alternative embodiments, the compound may comprise the formula X₁-X₂-X₃-X₄-X₅-X₆-X₇ (SEQ ID NO. 17), wherein X₇ is one or more linking amino acid residues comprising alanine wherein the linking residues join two sextapeptide compounds to each other, each sextapeptide having the sequence X₁-X₂-X₃-X₄-X₅-X₆, wherein X₁ is V or I or a mimetic of V or I; X₂ is G, or a mimetic of G; X₃ is A or L, or a mimetic of A or L; X₄ is M or S, or a mimetic of M or S, X₅ is P or a mimetic of P and X₆ is G or a mimetic of G.

A further embodiment relates to the use of peptide mimetics of GXXPG (SEQ ID NO. 2) peptides. In one embodiment, the peptides are modified to produce peptide mimetics by replacement of one or more naturally occurring side chains of the 20 genetically encoded amino acids (or D amino acids) with other side chains, for instance with groups such as alkyl, lower alkyl, cyclic 4-, 5-, 6-, to 7 membered alkyl, amide, amide lower alkyl, amide di (lower alkyl), lower alkoxy, hydroxy, carboxy and the lower ester derivatives thereof, and with 4-, 5-, 6-, to 7 membered heterocyclics. For example, proline analogs can be made in which the ring size of the proline residue is changed from 5 members to 4, 6, or 7 members. Cyclic groups can be saturated or unsaturated, and if unsaturated, can be aromatic or nonaromatic. Heterocyclic groups can contain one or more nitrogen, oxygen, and/or sulphur heteroatoms. Examples of such groups include the furazanyl, furyl, imidazolidinyl, imidazolyl, imidazolinyl, isothiazolyl, isoxazolyl, morpholinyl (e.g. morpholino), oxazolyl, piperazinyl (e.g. 1-piperazinyl), piperidyl (e.g. 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g. 1-pyrrolidinyl), pyrrolinyl, pyrrolyl, thiadiazolyl, thiazolyl, thienyl, thiomorpholinyl (e.g. thiomorpholino), and triazolyl. These heterocyclic groups can be substituted or unsubstituted. Where a group is substituted, the substituent can be alkyl, alkoxy, halogen, oxygen, or substituted or unsubstituted phenyl. Peptidomimetics may also have amino acid residues that have been chemically modified by phosphorylation, sulfonation, biotinylation, or the addition or removal of other moieties.

A variety of techniques are available for constructing peptide mimetics with the same or similar desired biological activity as the corresponding native but with more favorable activity than the peptide with respect to solubility, stability, and/or susceptibility to hydrolysis or proteolysis. See, e.g., Morgan & Gainor, Ann. Rep. Med. Chem. 24:243-252 (1989). Certain peptidomimetic compounds are based upon the amino acid sequence of the peptides of the invention. Often, peptidomimetic compounds are synthetic compounds having a three-dimensional structure (i.e. a “peptide motif”) based upon the three-dimensional structure of a selected peptide. The peptide motif provides the peptidomimetic compound with the desired biological activity, i.e., binding to PIF receptors, wherein the binding activity of the mimetic compound is not substantially reduced, and is often the same as or greater than the activity of the native peptide on which the mimetic is modeled. Peptidomimetic compounds can have additional characteristics that enhance their therapeutic application, such as increased cell permeability, greater affinity and/or avidity and prolonged biological half-life.

Peptidomimetic design strategies are readily available in the art. See, e.g., Ripka & Rich, Curr. Op. Chem. Biol. 2:441-452 (1998); Hruby et al., Curr. Op. Chem. Biol. 1:114-119 (1997); Hruby & Balse, Curr. Med. Chem. 9:945-970 (2000). One class of peptidomimetics a backbone that is partially or completely non-peptide, but mimics the peptide backbone atom-for atom and comprises side groups that likewise mimic the functionality of the side groups of the native amino acid residues. Several types of chemical bonds, e.g., ester, thioester, thioamide, retroamide, reduced carbonyl, dimethylene and ketomethylene bonds, are known in the art to be generally useful substitutes for peptide bonds in the construction of protease-resistant peptidomimetics. Another class of peptidomimetics comprises a small non-peptide molecule that binds to another peptide or protein, but which is not necessarily a structural mimetic of the native peptide. Yet another class of peptidomimetics has arisen from combinatorial chemistry and the generation of massive chemical libraries. These generally comprise novel templates which, though structurally unrelated to the native peptide, possess necessary functional groups positioned on a nonpeptide scaffold to serve as “topographical” mimetics of the original peptide (Ripka & Rich, 1998, supra).

In embodiments, the method may further comprise administering an agent selected from epitopes, cytokines, growth factors and combinations thereof. In embodiments, the method may further comprise administering an agent selected from epitopes, cytokines, growth factors, elastogenic peptides and combinations thereof. In embodiments, the method may further comprise administering retinoic acid.

Although the present invention has been described in considerable detail with reference to certain preferred embodiments thereof, other versions are possible. Therefore, the spirit and scope of the appended claims should not be limited to the description and the preferred embodiments disclosed herein.

### EXAMPLES

### Example 1

Cell culture products, including Dulbecco's modified Eagle's medium (DMEM), alpha minimal essential medium (αMEM), fetal bovine serum (FBS), 0.2 percent trypsin-0.02 percent EDTA and all other products came from GIBCO Life Technologies (Burlington, ON). The polyclonal antibody to tropoelastin was purchased from Elastin Products (Owensville, Mo.). The antibody-recognizing AKAAAKAAAKA (SEQ ID NO. 15) sequence on soluble tropoelastin was a generous gift from Dr. Barry Starcher of the University of Texas. The polyclonal anti-S-Gal antibody was used to detect the 67 kDa EBP. Immobilized pepsin and protein A for preparation of anti-S-Gal F(ab′)₂ fragments were purchased from Pierce Chemical Company (Rockford, Ill.). The Fluor X Fluorescent Dye kit for labeling the anti-S-Gal F(ab′)₂ was obtained from Research Organics, Inc. (Cleveland, Ohio). Monoclonal antibody to total chondroitin sulphate was purchased from Sigma (St. Louis, Mo.), and monoclonal antibody recognizing chondroitin-6-sulfate was obtained from Seikagaku Corporation (Tokyo, Japan). Monoclonal antibody to fibrillin was purchased from Biomeda Corp. (Foster City, Calif.). Monoclonal antibody to FKBP65 was purchased from BD Biosciences (San Jose, Calif.). Monoclonal antibody to collagen type I was purchased from Chemicon International (Temecula, Calif.). Monoclonal antibody to Hsp47 was purchased from Stressgen Bioreagents (Victoria, BC). Monoclonal antibody to fibronectin was purchased from Sigma. The secondary antibodies fluorescein-conjugated goat anti-rabbit (GAR-FITC) and goat anti-mouse (GAM-FITC) were purchased from Sigma, and rhodamine-conjugated goat anti-rabbit (GAR-RITC) and goat anti-mouse (GAM-RITC) were purchased from Chemicon International. DAPI and propidium iodide were purchased from Sigma. The DNeasy Tissue System for DNA assay and the RNeasy Mini Kit for isolation of total RNA were purchased from Qiagen (Mississauga, ON). The Superscript First-Strand Synthesis System for RT-PCR was purchased from Invitrogen Life Technologies (Carlsbad, Calif.). The OneStep RT-PCR Kit was purchased from Qiagen. The Enhanced Chemiluminescence (ECL) Detection Kit and the radio-labeled reagents [³H]-valine and [³H]-thymidine were purchased from Amersham Canada Ltd. (Oakville, ON), and the human GAPDH control was purchased from Clontech (Palo Alto, Calif.).

Cell cultures: With parental consent and Institutional Ethics Committee approval, SMCs were propagated from small aortic fragments obtained during the autopsy of a twelve-year-old male patient who died in a traffic accident. Fibroblasts derived from skin biopsies of three normal boys (4212, a one-year-old, 4184, a three-year-old, and 8972, a six-year-old) and three male patients with CS (OMIM#218040) (7669, a three-month-old, 9951, a one-year-old, and 10595, a two-year-old) were obtained from the cell repository of The Hospital for Sick Children in Toronto. All children who donated cultured cells were previously diagnosed and their clinical diagnoses were confirmed by genetic tests. Fibroblasts were also isolated from biopsies of the stretch-marked skin of three female patients, a 26-year-old (case 1), a 28-year-old (case 2) and a 36-year-old (case 3). All mentioned cells, initially stored at passage 2, were trypsinized and further maintained in αMEM supplemented with 10 percent FBS and 1 percent antibiotics/antimycotics. All experiments were performed on cells from passages 3-5. In experiments aimed at assessing ECM production, initially normal aortic SMCs, normal skin fibroblasts, and fibroblasts derived from stretch-marked skin patients (1×10⁵ cells/dish) were plated and maintained them at 37° C. for 48 hours; then, parallel confluent cultures were kept in separate incubators at 37, 39, or 41° C. Dermal fibroblasts derived from CS patients were plated more densely (2×10⁵ cells/dish) for immediate confluency; two hours after plating, parallel cultures were kept at 37, 39, or 41° C. for different periods of time, as indicated in the figure legends. All experiments were performed three times, and quadruplicate cultures from each experiment were used in each assay at the given time.

Immunostaining: At the end of the indicated incubation period (in), parallel cultures maintained at 37, 39, or 41° C. were washed and fixed in cold 100 percent methanol at −20° C. for 30 minutes. After one-hour-long blocking with 1 percent normal goat serum in PBS, cultures were then incubated for one hour (at room temperature) with antibodies recognizing ECM components (elastin, collagen I, fibronectin, and chondroitin sulfate) or antibodies recognizing the intracellular protein chaperones Hsp47, FKBP65 and S-Gal/EBP. All cultures were washed and then incubated for an additional hour with the appropriate (green) fluorescein-conjugated goat anti-rabbit (GAR-FITC) or goat anti-mouse (GAM-FITC) secondary antibodies. Nuclei were counterstained with (red) propidium iodide. For double immunostaining, parallel cultures were incubated simultaneously with pairs of monoclonal (anti-FKBP65, anti-Hsp47, or anti-EBP) and polyclonal (anti-tropoelastin) primary antibodies, followed by secondary GAM-RITC and GAR-FITC. The combination of polyclonal anti-S-Gal antibody (detecting EBP) and monoclonal anti-tropoelastin antibodies was also used to visualize the association of these two proteins. The nuclei were counterstained with blue DAPI. All immunostained cultures were then mounted in Elvanol and examined with a Nikon Eclipse E1000 microscope equipped with a source of fluorescent light and multiple filters. The images were obtained with a cooled CCD camera (Retiga EX, Qimaging, Surrey, BC). The morphometric analysis was then performed using Image-Pro Plus software from Media Cybernetics (Silver Spring, Md.).

Organ cultures of explants derived from human skin: Fragments of normal and stretch-marked skin collected during plastic surgery procedures from the stretch-marked skin of three female patients, cases 1, 2, and 3, were cut into multiple 4 mm² pieces, placed in culture dishes, and maintained for five days at 37 or 41° C. in DMEM culture medium containing 5 percent FBS. At the end of this period, all organ cultures were fixed in 1 percent buffered formalin, and their transverse serial histological sections were stained with Movat's pentachrome which shows elastin as black, glycosaminoglycans as green, collagen as yellow, and cell nuclei as dark blue. Previous studies have confirmed that the distribution of black-stained material with Movat's method entirely overlaps with immunodetectable elastin.

Monitoring S-Gal/EBP recycling kinetics: Human aortic SMCs were plated on cover slips in 35 mm dishes and grown to subconfluency. Multiple cultures were then preincubated for one hour with heat-inactivated 3 percent normal rabbit serum, 3 percent normal goat serum, and 1 percent BSA in order to block the Fc receptors and other possible nonspecific binding sites, then placed on ice, at 4° C. (to inhibit intracellular trafficking, including endocytosis). Cultures were then washed with cold PBS and externally labeled for 30 minutes with 1 μg/ml of F(ab′)₂ fragments of anti-S-Gal immunoglobulin directly conjugated to carboxyfluorescein. At the end of a 30-minute labeling period, the cultures were washed again in PBS and incubated at either to 37 or 41° C. for different time periods (5, 15, 25, 35, 45, and 55 minutes). After each chase period, cultures were extensively washed in medium containing 0.2 percent sodium azide, permeabilized with 100 percent methanol at −20° C., and washed again in PBS containing 1 percent BSA. The nuclei were then counterstained with DAPI.

To confirm that fluorescein-conjugated anti-S-Gal F(ab′)₂ binds specifically to the cell surface EBP, parallel control cultures were incubated for 15 minutes with an excess (5 μg/ml) of nonlabeled anti-S-Gal F(ab′)₂ or with a high concentration (1 mg/ml) of K-elastin, a ligand which binds to the same domain of the EBP as the anti-S-Gal antibody before incubation with labeled immunoglobulin. Also, additional control cultures were preincubated for 15 minutes with 0.1 M lactose, which releases EBP from the cell surface before exposure to fluorescein-labeled anti-S-Gal F(ab′)₂. Anti-S-Gal does not cross-react with elastin or with the catalytically active form of β-galactosidase.

Metabolic labeling and assays of tropoelastin and insoluble elastin: Aortic SMCs isolated from normal individual and skin fibroblasts derived from normal individuals and WBS and CS patients were initially plated at 1×10⁶ cells/35 mm dish and grown to confluency for two days in full DMEM with 10 percent FBS. The quadruplicate cultures were then incubated at 37, 39, or 41° C. for the time periods indicated in the figure legends in the presence of 1 [μCi/ml [³H]-valine. The pulse and chase experiments were also performed in cultures of dermal fibroblasts derived from CS patients. At the end of the incubation period, we assessed the levels of (metabolically labeled) soluble tropoelastin and insoluble elastin. Briefly, conditioned media were collected and immunoprecipitated with antibody that recognized the AKAAAKAAAKA (SEQ ID NO. 15) sequence on soluble tropoelastin. The soluble proteins still present in the intracellular compartments were first extracted with 0.1 mol/L of acetic acid and tropoelastin and were then immunoprecipitated from these extracts. The remaining cell layers containing the newly deposited extracellular matrix were scraped and boiled for 45 minutes in 0.5 ml of 0.1 N NaOH to solubilize all matrix components except elastin. The resulting pellets containing the insoluble elastin were then solubilized by boiling in 5.7 N HCl. The levels of the [³H]-valine-labeled, immunoprecipitable tropoelastin and insoluble elastin were then quantitatively assessed by scintillation counting and normalized per DNA content in each individual culture. The levels of metabolically labeled insoluble elastin were also assessed in cultured dermal explants.

Assessment of tropoelastin and S-Gal/EBP levels on message and protein levels: Human aortic SMCs and dermal fibroblasts isolated from normal and stretch-marked skin fragments, as well as dermal fibroblasts derived from WBS and CS patients, were incubated at 37 or 41° C. for the different time periods indicated in the figure legends. Total RNA was isolated using the RNeasy Mini Kit according to manufacturer's instructions. Elastin and EBP mRNA levels were examined using Qiagen One Step RT-PCR, where reverse transcription and PCR were carried out sequentially according to the manufacturer's instructions with the following primers: human elastin, the sense primer 5′-GGTGCGGTGGTTCCTCAGCCTGG-3′ (SEQ ID NO. 18), the antisense primer 5′-GGGCCTTGAGATACCCCAGTG-3′ (SEQ ID NO. 19), the human spliced variant of (3-galactosidase (EBP), the sense primer 5′-GTTCCTGGTTCGCATCCTCCT-3′ (SEQ ID NO. 20), and the antisense primer 5′-GCCAGGTAATGTCTGGATGGC-3′ (SEQ ID NO. 21). In each experiment, we added 0.5 μg of total RNA to each one-step RT-PCR (Qiagen One-Step RT-PCR Kit), and reactions were set up according to the manufacturer's instructions in a total volume of 25 μl. The amounts of tropoelastin and S-Gal/EBP mRNAs detected in each sample were normalized to the amount of GAPDH mRNA. The levels of tropoelastin and S-Gal/EBP proteins were assessed by western blots with the respective specific antibodies and standardized to the β-actin levels.

Metabolic labeling with [³⁵S]-sulfate and assessment of sulphated glycosaminoglycan production: Skin fibroblasts derived from three normal individuals and three CS patients were initially plated at 50,000 cells/dish in normal medium in 35 mm culture dishes immediately after trypsinization. Two hours later, the medium was changed to sulphate-free DMEM containing 2 [μCi/ml of [³⁵S]-sulfate, and the parallel quadruplicate cultures were incubated at 37 and 41° C. for 24 hours. At the end of the incubation period, the cultures were extensively washed in PBS and then digested at 60° C. for two hours with proteinase K (250 μg/ml). The cultures were then rinsed with ammonium acetate and incubated for another 20 minutes at 100° C. to inactivate the proteinase K. This step was followed by precipitation with 100 percent ethanol at −20° C. The precipitates were finally lyophilized and dissolved in water, and the content of the newly produced sulphated glycosaminoglycans were immunoprecipitated with antibodies recognizing total chondroitin sulfate and chondroitin-6-sulfate. The obtained products were then quantitatively assessed by scintillation counting and normalized per DNA content in each individual culture.

In all studies, quadruplicate samples in each experimental group were assayed in at least three separate experiments. The mean and standard deviation (SD) for each group were calculated and the statistical analyses were carried out. Unpaired Student's t-tests were performed, and the statistical significance was shown. P-values less than 0.05 (p<0.05) were considered significant.

### Example 2

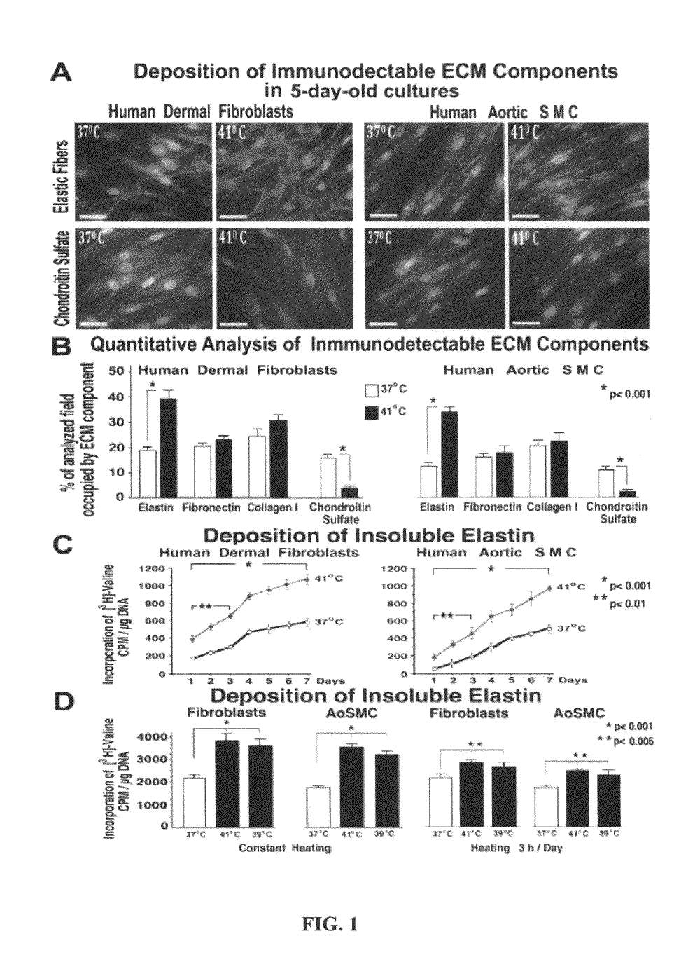

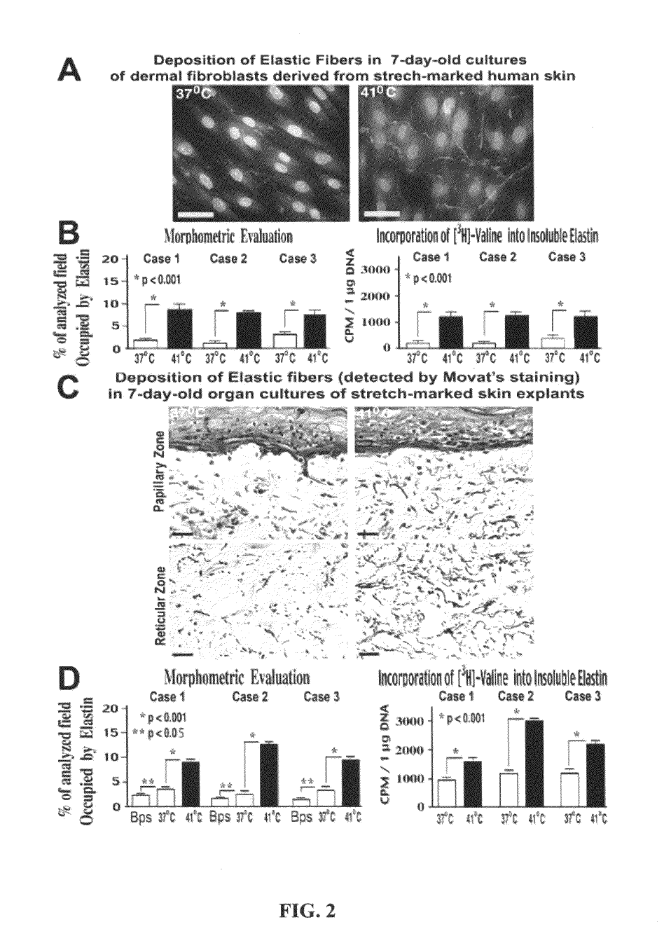

Cultured cells exposed to hyperthermia produce more elastic fibers than cultures maintained at 37° C.: The results of initial experiments examining the effect of hyperthermia on the production of elastic fibers demonstrated that cultures of normal human skin fibroblasts and normal human aortic SMCs maintained for five days at 41° C. produced more immunodetected elastic fibers than cultures kept at 37° C. Remarkably, all cultures exposed to 41° C. demonstrated a significant decrease in the level of chondroitin-sulfate-containing moieties (FIG. 1A). Morphometric analysis of multiple parallel cultures immunostained with specified antibodies further indicated that hyperthermia exclusively upregulated the deposition of elastic fibers by an average of 94 to 112 percent and downregulated the deposition of chondroitin-sulfate-containing moieties by an average of 82 to 88 percent. Meaningfully, exposure to hyperthermia did not affect the levels of other fibrous ECM components, such as collagen type I or fibronectin (FIG. 1B). The significant and selective increase in the deposition of immunodetectable elastic fibers in hyperthermia-exposed cultures was further validated by the results of the quantitative biochemical assay of (cross-linked) insoluble elastin after the metabolic labeling of cultured cells with [³H]-valine. The time course observations indicated that the hyperthermia-induced enhancement in the deposition of radioactive elastin was evident as early as day one and steadily increased until day seven (FIG. 1C). Moreover, a seven-day-long exposure of cultured cells to only 39° C. also induced a significant upregulation in the net deposition of [³H]-valine-labeled insoluble elastin. The observed values were only insignificantly lower than the values noted in cultures kept at 41° C. Of practical importance, additional results showed that the cultures of both cell types exposed to 41 or 39° C. for only three hours a day also demonstrated a significant increase in the net deposition of insoluble elastin (assessed in seven-day-old cultures), as compared to control cultures kept at 37° C. (FIG. 1D). Using the same techniques, it was also documented that seven days of exposure to hyperthermia induced production of new elastic fibers in monolayer cultures of dermal fibroblasts isolated from biopsies of the stretch-marked skin of three female patients (cases 1, 2, and 3 (FIGS. 2A and B). It was then demonstrated that hyperthermia also induced production of long elastic fibers in organ cultures of skin explants derived from these patients (FIGS. 2C and D). In contrast, the parallel explants (derived from the same initial biopsies) maintained at 37° C. revealed only thin and short elastic fibers. It is noteworthy that histological sections of skin explants exposed to 41° C. did not reveal any accumulation of elastotic material as has been reported in skin exposed to 43° C. (31-33). The heightened number of elastic fibers in explants maintained for seven days at 41° C. (morphometric evaluation revealed a two- to threefold increase) were additionally validated by proportionally higher values of metabolically labeled insoluble elastin detected in parallel cultures. Jointly, both methods indicated that dermal explants of stretch-marked skin produced significantly more new elastic fibers when cultured at 41° C. Since the number and size of elastic fibers observed in sections of explants maintained for seven days at 37° C. also exceeded values observed in the initial biopsies evaluated at time zero (FIG. 2D), the possibility of elastolysis in explants cultured at 37° C. was excluded.

### Example 3

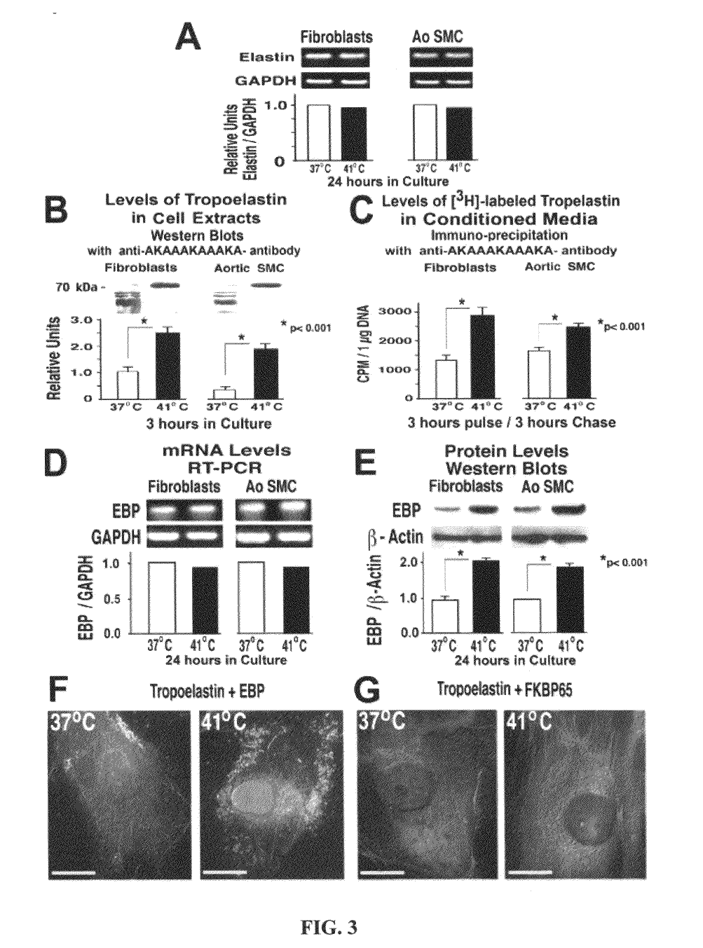

Hyperthermia does not affect the steady-state level of tropoelastin mRNA but improves the durability of the newly translated elastin. In an attempt to determine the mechanism by which hyperthermia stimulates an increase in net elastin deposition in cultures of dermal fibroblasts and aortic SMCs, the steady-state levels of tropoelastin mRNA in cultures maintained at 37° C. and 41° C. for 24 hours were compared. The results of quantitative RT-PCR analysis showed that exposure to hyperthermia did not affect elastin mRNA levels (FIG. 3A). On the other hand, western blot analysis using an antibody recognizing the AKAAAKAAAAAKA (SEQ ID NO. 15) sequence (exposed only in soluble tropoelastin and hidden in polymerized elastin) detected more full-length (72 kDa) tropoelastin and less lower molecular weight tropoelastin degradation products in extracts of dermal fibroblasts and aortic SMCs exposed to 41° C. for only three hours, as compared to their counterparts kept at 37° C. (FIG. 3B). Since RT-PCR analysis demonstrated no change in tropoelastin mRNA levels during a three-hour exposure to 41° C. (data not shown), these results suggest that hyperthermia improves the durability of newly translated tropoelastin. Furthermore, it was demonstrated that dermal fibroblasts and aortic SMCs (pulsed for three hours with [³H]-valine) that were exposed to 41° C. secreted significantly more [³H]-labeled tropoelastin that was immunoprecipitated with anti-AKAAAKAAAKA (SEQ ID NO. 15) antibody from the conditioned media following a three-hour chase than cultures kept at 37° C. (FIG. 3C). Jointly, the presented results indicate that, in addition to preserving newly produced tropoelastin better, hyperthermia also stimulates secretion of this precursor protein.

### Example 4

Hyperthermia-exposed cells demonstrate heightened levels of intracellular S-Gal/EBP. Although cells exposed to hyperthermia did not demonstrate any increase in the steady-state levels of S-Gal/EBP mRNA (FIG. 3D), they revealed increased levels of this tropoelastin chaperone detected by western blotting (FIG. 3E). This suggested that the heightened levels of S-Gal/EBP detected in hyperthermia-exposed cells did not result from the increased synthesis of this protein but likely reflected its intracellular retention. Moreover, a double immunostaining using antibodies against tropoelastin and S-Gal/EBP clearly documented a peculiar pattern of association between intracellular tropoelastin and its chaperone in hyperthermia-exposed cells. It was demonstrated that, in aortic SMCs maintained at 37° C., the majority of tropoelastin (green fluorescence) was detected in the endoplasmic reticulum apart from the S-Gal/EBP (red fluorescence) and that colocalization of these two proteins (yellow fluorescence) occurred only in the peripheral vesicular compartment. In contrast, cells maintained at 41° C. demonstrated that the bulk of the intracellular tropoelastin colocalized with S-Gal/EBP (yellow fluorescence) in a large endosomal compartment localized in the perinuclear region and in numerous peripheral vesicles (FIG. 3F).

In addition, the possible involvement of two other molecular chaperones in the hyperthermia-induced preservation of intracellular elastin were investigated. These experiments produced negative results. It was found that neither Hsp47, a known molecular chaperone for such proline-rich proteins as collagen nor FKBP65, previously shown to colocalize with tropoelastin in the distended endoplasmic reticulum of brefeldin-treated cells, would interact with tropoelastin. Western blot analysis and immunostaining indicated that, while hyperthermia upregulated the levels of Hsp47 in both tested cell types, it did not influence basal levels of immunodetectable FKBP65 (data not shown). The double immunostaining of cultured dermal fibroblasts and aortic SMCs maintained at 37 or 41° C. also indicated that neither Hsp47 (data not shown) nor FKBP65 (FIG. 3G) colocalized with intracellular or extracellular elastin.

### Example 5

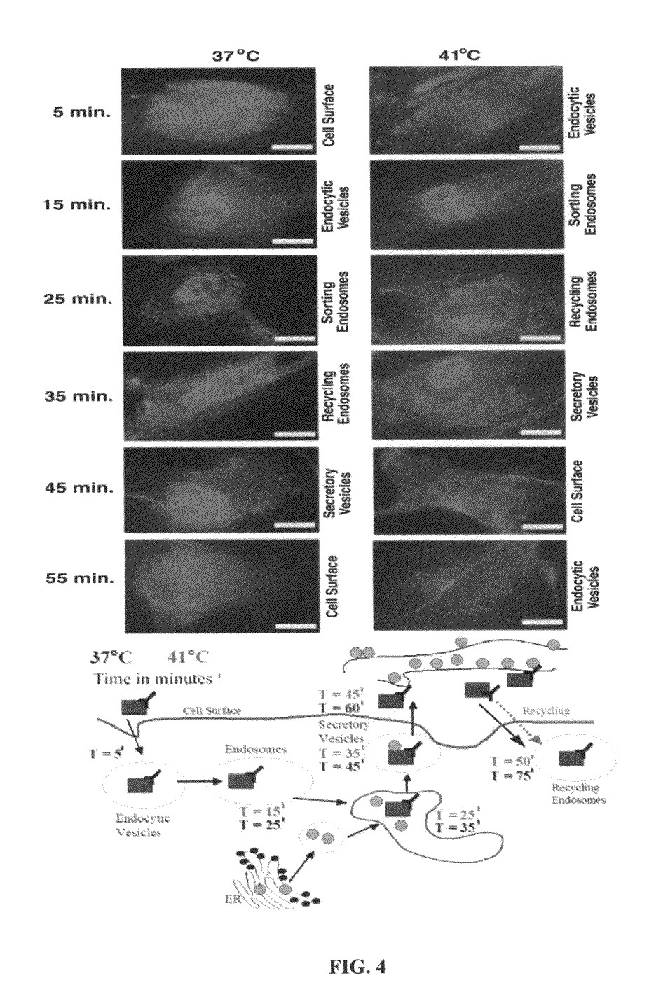

Hyperthermia induces faster recycling of the S-Gal/EBP. S-Gal/EBP acts as a recyclable chaperone that delivers multiple tropoelastin molecules to the cell surface in several consecutive rounds. It was therefore examined whether hyperthermia might affect the intracellular trafficking of S-Gal/EBP and whether the kinetics of its recycling would potentially improve the net secretion of tropoelastin in aortic SMCs. In order to trace EBP trafficking, multiple subconfluent cultures of aortic SMCs were cooled to 4° C. (to inhibit intracellular trafficking, including endocytosis), externally labeled with anti-S-Gal antibody for 15 minutes, and then transferred to incubate at either 37 or 41° C. for different periods (5, 15, 25, 35, 45 and 55 minutes). Using both regular fluorescence microscopy and confocal microscopy, exposing aortic SMCs to anti-S-Gal antibody at 4° C. for 15 minutes resulted in the exclusive labeling of cell-surface EBP molecules. The present experiments, which monitored aortic SMCs by fluorescence microscopy, demonstrated that, five minutes after the transfer of cultures to 37° C., the anti-S-Gal/EBP complexes were still detected on the cell surface; but, during the next ten minutes, they began an internalization that could be clearly detected in the endocytic vesicles and in the endosomal cisternas localized in the cell center 15 minutes after the cell transfer to 37° C. Approximately ten minutes later, the anti-S-Gal-labeled EBP molecules were concentrated mostly in the perinuclear endosomal compartments, and following the next three ten-minute intervals, the anti-S-Gal/EBP complexes were present in the peripheral endosomal cisternas, in exocytic vesicles, and again at the cell surface (FIG. 4, left panels). Importantly, it was demonstrate that the overall S-Gal/EBP recycling process occurred significantly faster in cells maintained at 41° C., resulting in a second round of its recycling 55 minutes after the transfer of cells to 41° C. (FIG. 4, right panels).

### Example 6

Hyperthermia induces inhibition of chondroitin-6-sulfate accumulation in CS-derived fibroblasts, thereby restoring normal elastogenesis. Since normal dermal fibroblasts exposed to 41° C. produce fewer chondroitin sulfate moieties (FIG. 1A) and contain more S-Gal/EBP than counterparts maintained at 37° C. (FIGS. 2E and G), it was tested that whether hyperthermia might also inhibit the synthesis of glycosaminoglycans containing chondroitin-6-sulfate in CS-derived fibroblasts, which were characterized by the selective accumulation of this glycosaminoglycan, which triggers the shedding of S-Gal/EBP.

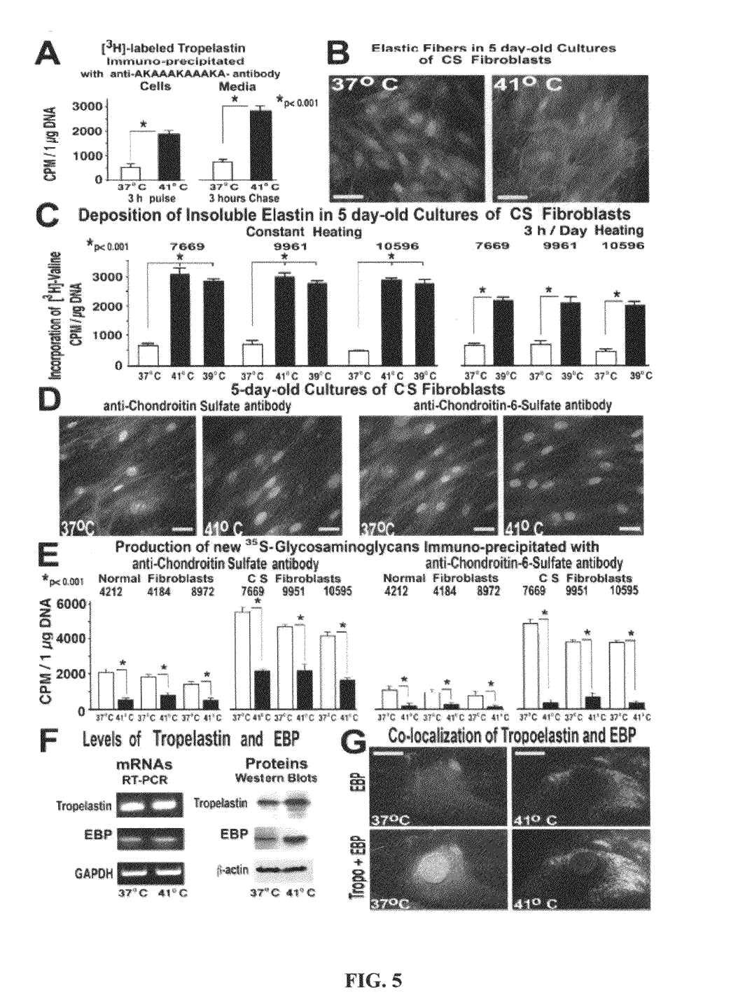

Indeed, it was demonstrated that CS fibroblasts exposed to hyperthermia (just two hours after plating into the secondary cultures) exhibited heightened levels of intracellular tropoelastin, higher rates of its secretion, and much higher ultimate deposition of elastic fibers than their counterparts maintained at 37° C. (FIG. 5A-C). Using immunostaining with anti-chondroitin sulfate and with anti-chondroitin-6-sulfate antibodies, it was also documented that exposure to hyperthermia rectified the peculiar accumulation of these sulphated glycosaminoglycans that has been consistently observed in parallel cultures of CS-derived dermal fibroblasts maintained at 37° C. (FIG. 5D). The results of further experiments clearly indicated that hyperthermia significantly inhibited the new synthesis of [³⁵S]-sulphate-labeled glycosaminoglycans that could be immunoprecipitated from extracts of normal fibroblasts and CS-derived fibroblasts using antibodies that recognize total chondroitin sulfate and chondroitin-6-sulfate (FIG. 4E). Importantly, it was also demonstrated that the hyperthermia-dependent inhibition of the deposition of chondroitin-6-sulfate by cultured CS fibroblasts coincided with a marked increase in cell-associated S-Gal/EBP, as immunodetected by western blotting (FIG. 5F). Moreover, double immunostaining (FIG. 5G) further demonstrated that hyperthermia-exposed CS fibroblasts contained more secretory vesicles (yellow fluorescence), in which the immunodetected EBP (red fluorescence) colocalized with the immunodetected tropoelastin (green fluorescence). In addition, a similar range in the inhibition of the deposition of immunodetectable and immunoprecipitable [³⁵S]-sulphate-labeled chondroitin sulfates in all tested cell types after exposure to 39° C. (data not shown) were observed.

1. A method of improving the appearance of a tissue site in a subject in need thereof comprising applying heat to the tissue site.

2. The method of claim 1, wherein heat comprises a temperature of about 39° C. to about 41° C.

3. The method of claim 1, wherein the temperature is selected from the group consisting of about 39° C., about 39.5° C., about 40° C., about 40.5° C. and about 41° C.

4. The method of claim 1, wherein the tissue site comprises scar tissue, visible lines, wrinkles and combinations thereof.

5. The method of claim 1, wherein net deposition of chondroitin-sulfate containing moieties is inhibited.

6. The method of claim 1, wherein the subject suffers from elastinopathy.

7. The method of claim 6, wherein the elastinopathy comprises Marfan syndrome; autosomal dominant Weill-Marchesani syndrome; severe neonatal Marfan syndrome; dominant ectopia lentis; isolated skeletal features of Marfan syndrome; Beals syndrome; Familial mitral valve prolapse syndrome (MVP); mitral valve prolapse, myopia, minimal or no aortic dilation, subtle skeletal changes and skin changes (MASS phenotype); Shprintzen-Goldberg syndrome; supravalvular aortic stenosis; Williams syndrome; Costello syndrome, Loeys-Dietz syndrome and Cutis laxa.

8. The method of claim 1 further comprising administering an effective amount of an elastogenic peptide.

9. The method of claim 8, wherein the elastogenic peptide is selected from VGVAPG (SEQ ID NO. 1), VGAMPG (SEQ ID NO. 4), VGLSPG (SEQ ID NO. 5), IGAMPG (SEQ ID NO. 6), IGLSPG (SEQ ID NO. 7), VGAMPGAAAAAVGAMPG (SEQ ID NO. 8), VGLSPGAAAAAVGLSPG (SEQ ID NO. 9), VGVAPGAAAAAVGVAPG (SEQ ID NO. 10), IGAMPGAAAAAIGAMPG (SEQ ID NO. 11), IGLSPGAAAAAIGLSPG (SEQ ID NO. 12), IGVAPG (SEQ ID NO. 13), IGVAPGAAAAAIGVAPG (SEQ ID NO. 14) and combinations thereof.

10. The method of claim 8, wherein the elastogenic peptide is administered by injection or topical administration.

11. The method of claim 1, wherein the tissue site comprises connective tissue.

12. The method of claim 11, wherein the tissue site is selected from the group consisting of dermal fibroblasts, smooth muscles cells, mouth tissue, hair follicles, and corneal tissue.

13. The method of claim 1, wherein hyperproliferative collagenous neointimal formation in arterial SMC is inhibited.

14. The method of claim 1, wherein the production of insoluble elastin in the tissue site to which it is administered is stimulated.

15. The method of claim 1, wherein the endogenous synthesis and deposition of elastin in the tissue site to which it is administered is stimulated.

16. The method of claim 1, wherein the deposition of collagen in the tissue site to which it is administered is stimulated.

17. The method of claim 1, wherein the appearance of the tissue site presenting visible lines or wrinkles is improved.

18. The method of claim 1, wherein the appearance of the tissue site comprising scar tissue is improved.

19. The method of claim 1 further comprising administering an agent selected from epitopes, cytokines, growth factors and combinations thereof.

20. The method of claim 1 further comprising administering retinoic acid.

21. The method of claim 1, wherein the production of collagen is stimulated.

22. The method of claim 1, wherein the heat is applied to the tissue site one to three times a day.

23. The method of claim 1, wherein the heat is applied to the tissue site for a period of about five minutes to about one hour.
